(19) United States
(12) Patent Application Publication
(10) Pub. No.: US 2015/0175998 A1
(43) Pub. Date: Jun. 25, 2015

(54) Methods and Kits to Create Protein Substrate˜HECT-Ubiquitin Ligase Pairs

(71) Applicant: Northwestern University, Evanston, IL (US)

(72) Inventors: Alexander Statsyuk, Evanston, IL (US); Sungjin Park, Evanston, IL (US); Neil L. Kelleher, Evanston, IL (US); Paul M. Thomas, Morton Grove, IL (US); Ioanna Ntai, Evanston, IL (US)

(21) Appl. No.: 14/416,055

(22) PCT Filed: Jul. 18, 2013
(86) PCT No.: PCT/US2013/051123

§ 371 Date: Jan. 20, 2015

Related U.S. Application Data
(60) Provisional application No. 61/674,152, filed on Jul. 20, 2012.

(51) Int. Cl. C12N 9/00; C12Q 1/00

(52) U.S. Cl. CPC C12N 9/93; C12Q 1/008; C12Y 603/02019; G01N 2333/9015

(57) ABSTRACT

Methods and kits to use in the isolation and identification of crosslinked protein substrate ubiquitin ligase complexes are disclosed. More specifically the methods and kits disclosed herein describe the use of bifunctional thiol-and-amine crosslinkers to covalently bind an endogenous or exogenous HECT-ubiquitin ligase to a downstream protein substrate, preferably in a cell lysate.

### CROSS-REFERENCE TO RELATED APPLICATIONS

This application claims benefit of priority under 35 U.S.C. 119 from U.S. Provisional Application No. 61/674,152, filed Jul. 20, 2012 and entitled “METHODS AND KITS TO CREATE PROTEIN SUBSTRATE˜HECT-UBIQUITIN LIGASE PAIRS,” the contents of which are incorporated by reference in its entirety.

### FIELD

Methods and kits to use in the identification of crosslinked protein substrate ubiquitin ligase complexes are disclosed. More specifically, the methods and kits disclosed herein describe the use of bifunctional thiol-and-amine crosslinkers to covalently bind endogenous or exogenous HECT-ubiquitin ligases to a downstream protein substrate, preferably in cell lysate.

### BACKGROUND

Protein ubiquitination is a reversible, enzymatic, posttranslational modification process that regulates signal transduction, transcription, and protein lifespan. More and more, misregulation of the ubiquitination cascade has been observed in a host of mammalian diseases including cancer, neurodegenerative disorders, and hypertensive disorders. For example, mutations and amplifications in HECT ubiquitination ligase (E3) genes have been demonstrated to cause neurodegenerative diseases (e.g. Angelman syndrome), hypertensive disorders (e.g., Liddle's syndrome), and cancers.

There are currently two known methods to isolate protein substrate HECT-ubiquitin ligase pairs. These include the in vitro assay and the immunoprecipitation assay. Each of these assays has its disadvantages. For example, the in vitro assay demonstrates particularity in its protein substrate HECT-ubiquitin ligase interactions, but is not conducted in cell lysate. Moreover, this assay requires the inefficient and time-consuming step of immobilizing candidate protein substrates onto proteome arrays before isolating the protein substrate HECT-ubiquitin ligase pairs. The immunoprecipitation assay, on the other hand, does not require the immobilization of candidate protein substrates onto arrays, and is run in cell lysate, but does not demonstrate particularity with respect to isolating protein substrate HECT-ubiquitin ligase pairs. For example, it is common to co-precipitate undesired ubiquitin ligase protein pairs along with the protein substrate HECT-ubiquitin ligase pairs of interest.

### SUMMARY

In a first aspect, a method for forming a crosslinked protein substrate˜HECT-ubiquitin ligase complex is disclosed herein. The method involves providing a buffer solution, adding a thiol-and-amine crosslinker to the buffer solution, reacting the resultant mixture to create a crosslinked protein substrate˜HECT-ubiquitin complex. The method includes an optional step of adding a quenching solution to the mixture.

In this aspect, the buffer solution may include a mammalian cell lysate. Alternatively, the buffer solution may include a HECT-ubiquitin ligase, such that specific interactions between a particular HECT-ubiquitin ligase and varying protein substrates can be investigated. The HECT-ubiquitin ligases that can be incorporated in the method include, but are not limited to: Rsp5; Ufd4; Hu15; Tom1; Hu14; NEDD4; NEDD4L; ITCH; WWP1; WWP2; SMURF1; SMURF2; NEDL1; NEDL2; E6AP; HECTD2; KIAA0614; TRIP12; G2E3; EDD; HACE1; HECTD1; UBE3B; UBEC; KIAA0317; HUWE1; HECTD3; HERC1; HERC2; HERC3; HERC4; HERC5; HERC6; SopA; and NIeL.

The thiol-and-amine crosslinkers that can be used in this aspect include, but are not limited to, compounds 10-16 disclosed in FIG. 3. The quenching solution may be include Laemmli loading buffer, β-mercaptoethanol, tris buffer, and combinations thereof.

In a second aspect, a kit for forming a crosslinked protein substrate˜HECT-ubiquitin ligase complex in cell lysate is disclosed herein. In this aspect, the kit includes a thiol-and-amine crosslinker and a set of instructions to use the thiol-and-amine crosslinker to crosslink an endogenous protein substrate with an endogenous HECT-ubiquitin ligase resulting in the formation of a crosslinked protein substrate˜HECT-ubiquitin ligase complex. In this aspect, the thiol-and-amine crosslinker that can be used in the kit include, but are not limited to, compounds 10-16 disclosed in FIG. 3.

In a third aspect, a kit for forming a crosslinked protein substrate˜HECT-ubiquitin ligase complex in a buffer solution is disclosed herein. The kit includes a buffer solution, a thiol-and-amine crosslinker, and instructions to use said thiol-and-amine crosslinker to crosslink a protein substrate with a HECT-ubiquitin ligase resulting in the formation of a crosslinked protein substrate˜HECT-ubiquitin ligase complex.

In this aspect, the buffer solution may include a mammalian cell lysate. Alternatively, the buffer solution may include a HECT-ubiquitin ligase, such that specific interactions between a particular HECT-ubiquitin ligase and varying protein substrates can be identified. The HECT-ubiquitin ligases that can be incorporated in the kit include, but are not limited to: Rsp5; Ufd4; Hu15; Tom1; Hu14; NEDD4; NEDD4L; ITCH; WWP1; WWP2; SMURF1; SMURF2; NEDL1; NEDL2; E6AP; HECTD2; KIAA0614; TRIP12; G2E3; EDD; HACE1; HECTD1; UBE3B; UBEC; KIAA0317; HUWE1; HECTD3; HERC1; HERC2; HERC3; HERC4; HERC5; HERC6; SopA; and NIeL. Finally, the thiol-and-amine crosslinkers that can be used in this method include, but are not limited to, compounds 10-16 disclosed in FIG. 3.

### BRIEF DESCRIPTION OF THE DRAWINGS

FIG. 1 depicts the normal protein substrate ubiquitination process.

FIG. 2 depicts the use of a thiol-and-amine crosslinker to isolate a downstream protein substrate of a HECT-ubiquitin ligase.

FIG. 3 illustrates SDS-PAGE data showing the dependence of forming a crosslinked protein substrate˜HECT-ubiqutin ligase complex in the absence (“none”) or presence of different thiol-and-amine crosslinkers (10, 11, 12, 13, 14, 15 and 16) with a test protein substrate (GFP-Sic60) and different forms of a yeast HECT-ubiquitin ligase (Rsp5, C777A, Δ3C and Δ4C).

FIG. 4 illustrates the domain structure of yeast HECT-ubiquitin ligase, Rsp5 (17), and mutant forms, Rsp5 C777A (18), Rsp5 Δ3C (19), and Rsp Δ4C (20).

FIG. 5 illustrates SDS-PAGE data demonstrating the dependence of forming a crosslinked protein substrate˜HECT-ubiquitin ligase complex (band at ˜130 kDa) upon a thiol-and-amine crosslinker (10) with a test protein substrate (GFP-Sic60) and a HECT-ubiquitin ligase (Rsp5) in the presence of a cell lysate (HeLa extract).

FIG. 6A depicts the domain structure for a HECT-ubiquitin ligase (Rsp5) and a test protein substrate (Sic60-GFP).

FIG. 6B illustrates SDS-PAGE data showing the ubiquitination cascade reaction that occurs using these molecules in the absence (“−”) or presence (“+”) of ATP.

FIG. 7A illustrates SDS-PAGE data demonstrating the dependence of forming a crosslinked protein substrate˜HECT-ubiqutin ligase complex (band at ˜130 kDa) upon a thiol-and-amine crosslinker (10) with a test protein substrate (Sic60-GFP) and a HECT-ubiquitin ligase (Rsp5) in the presence of a buffer.

FIG. 7B illustrates SDS-PAGE data of control reactions showing the lack of crosslinked complex formation with a yeast HECT-ubiquitin ligase (Rsp5) alone or with a test protein substrate (Sic60-GFP) alone in the absence (“−”) or presence (“+”) of a thiol-and-amine crosslinker (10).

FIG. 8A illustrates the domain structure of yeast HECT-ubiquitin ligase, Rsp5, and mutant forms, Rsp5 C777A, Rsp5 Δ3C, and Rsp Δ4C.

FIG. 8B illustrates SDS-PAGE data showing the ubiquitination cascade reaction that occurs using these molecules in the absence (“−”) or presence (“+”) of ATP.

FIG. 8C illustrates data showing the relative amount of crosslinked fluorescent product (band at ˜130 kDa) formed between test protein substrate Sic60-GFP and yeast HECT-ubiquitin ligase Rsp5 in the presence of crosslinker 10 in the absence (“−”) or presence (“+”) of HeLa cell lysate.

FIG. 9 illustrates the extent of crosslinking of Rsp5 HECT-ubiquitin ligase (lanes 1, 5) and of Rsp5 mutants C777A (lanes 2, 6), Δ3C (lanes 3, 7) and Δ4C (lanes 4, 8) with Sic60-GFP in the absence (lanes 1-4) or presence (lanes 5-8) of crosslinker 8 (disuccinimidyl glutarate).

FIG. 10A depicts the domain structure of the Rsp5 HECT-ubiquitin ligase and a modified mutant form of the Rsp5 HECT-ubiquitin ligase (Rsp5ΔWW).

FIG. 10B illustrates SDS-PAGE data for the ubiquitination activity of the Rsp5ΔWW mutant HECT-ubiquitin ligase with the test protein substrate Sic60-GFP in the absence (“−”) or presence (“+”) of ATP.

FIG. 11A depicts the domain structure of the Sic60-GFP test protein substrate and a modified mutant form of the Sic60-GFP test protein substrate (Sic60-GFPΔPY).

FIG. 11B illustrates SDS-PAGE data for the ubiquitination activity of Rsp5 with the Sic60-GFPΔPY test protein substrate in the absence (“−”) or presence (“+”) of ATP.

### DETAILED DESCRIPTION

The method disclosed herein is a novel approach to crosslink a protein substrate with a HECT-ubiquitin ligase so that protein substrates of HECT-ubiquitin ligases can be identified. This robust method allows one to identify protein substrates of HECT-ubiquitin ligases without having to immobilize candidate protein substrates onto proteome arrays. Moreover, the method disclosed herein solves the long-felt but unresolved need for identifying protein substrates of HECT-ubiquitin ligases in cell lysate without the additional isolation of protein substrates of non-HECT ubiquitin ligases. Finally, the method enables one to rapidly identify protein substrates of HECT-ubiquitin ligase important for use as reagents and markers for various purposes, including uses for diagnostic and therapeutic applications.

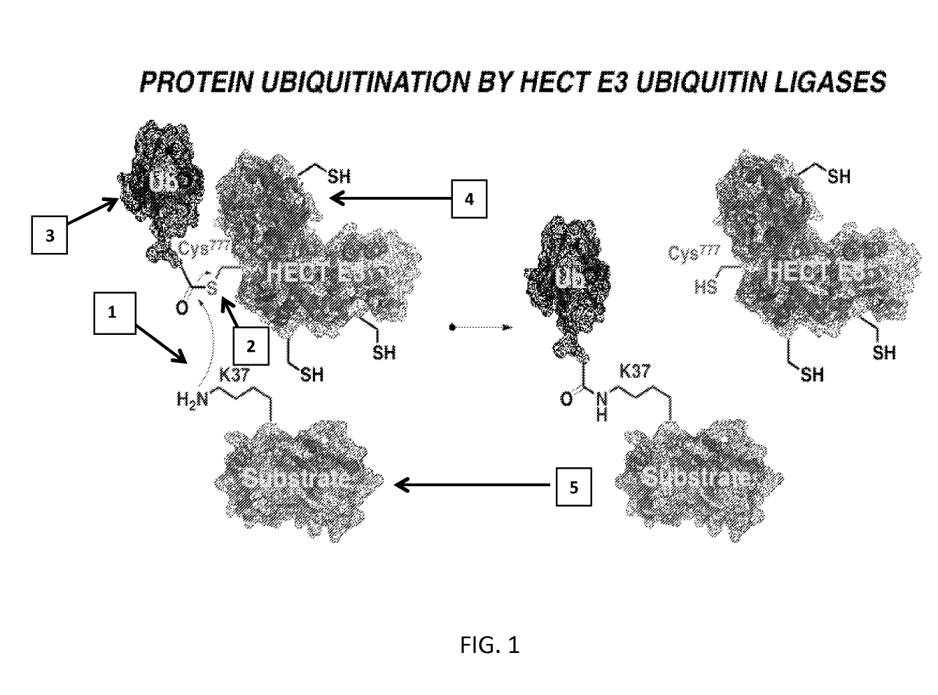

Referring to FIG. 1, in a normal ubiquitination cascade, a protein substrate site 1 comes into close proximity with a ubiquitin ligase active site 2. The physical proximity of the sites to each other in the ubiquitination process transfers ubiquitin 3 from the ubiquitin ligase molecule 4 to the protein substrate 5.

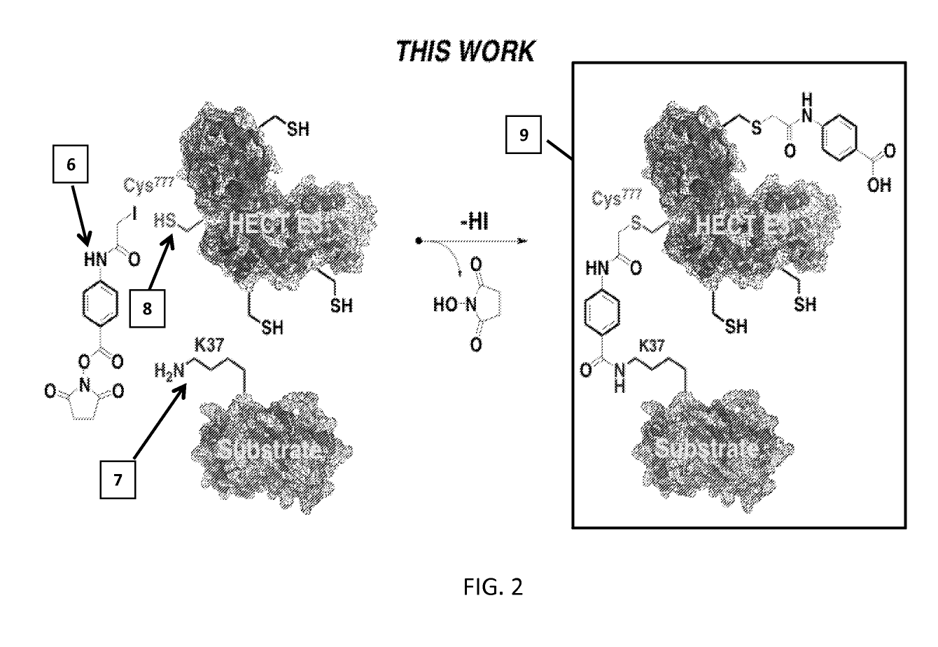

Referring to FIG. 2, in a first embodiment disclosed herein, a thiol-and-amine crosslinker 6 may be used to covalently crosslink the protein substrate site 7 with the HECT-ubiquitin ligase active site 8 to create a crosslinked protein substrate˜HECT-ubiquitin ligase complex in the form of a crosslinked protein substrate˜HECT-ubiquitin ligase pair 9. The crosslinked protein substrate˜HECT-ubiquitin ligase complex may be separated from non-crosslinked materials in a subsequent step.

A thiol-and-amine crosslinker used herein to crosslink a protein substrate with a HECT-ubiquitin ligase has a thiol reactive group. The thiol reactive group can be chosen from amongst any electrophilic moiety that interacts with cysteine, and can include preferably one moiety selected from the following group:

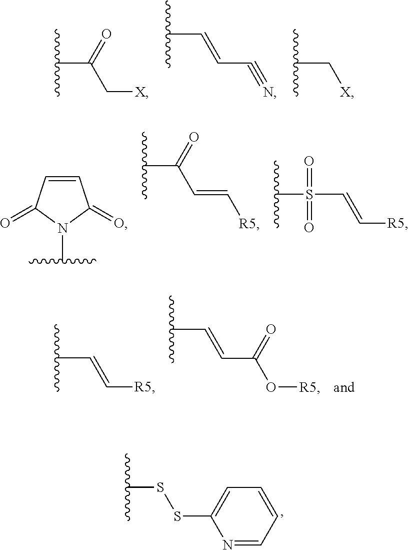

wherein X can be a halogen. Additionally, R5 can be chosen from the group including hydrogen, substituted alkyl, unsubstituted alkyl, substituted heteroalkyl, substituted cycloalkyl, unsubstituted cycloalkyl, substituted heterocycloalkyl, substituted heteroaryl, or unsubstituted heteroaryl.

The thiol-and-amine crosslinker disclosed herein to crosslink a protein substrate with a HECT-ubiquitin ligase also has an amine reactive group. The amine reactive group can be chosen from amongst any electrophilic moiety that interacts with lysine, and can include preferably one moiety selected from the following group:

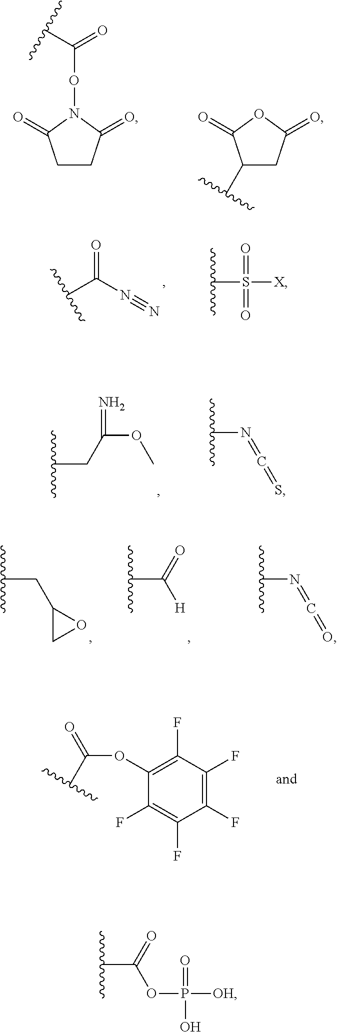

wherein X is a halogen.

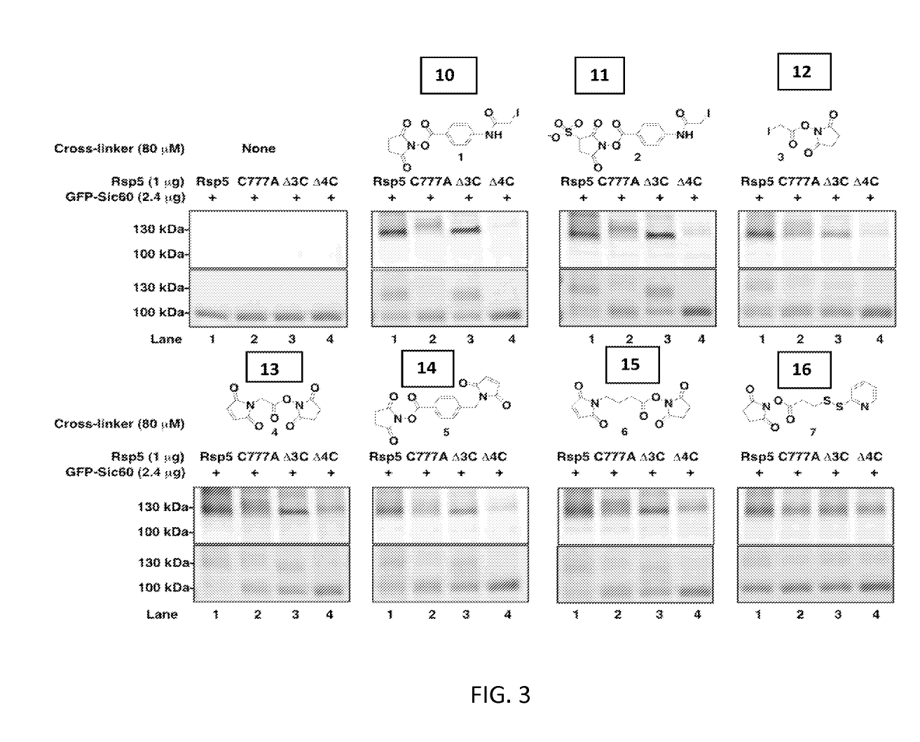

More specifically, and now referring to FIG. 3, in an embodiment disclosed herein, the thiol-and-amine crosslinker used herein may be chosen preferably from the following group of bifunctional crosslinking reagents, each of which includes a thiol reactive group and an amine reactive group as described previously: (10)

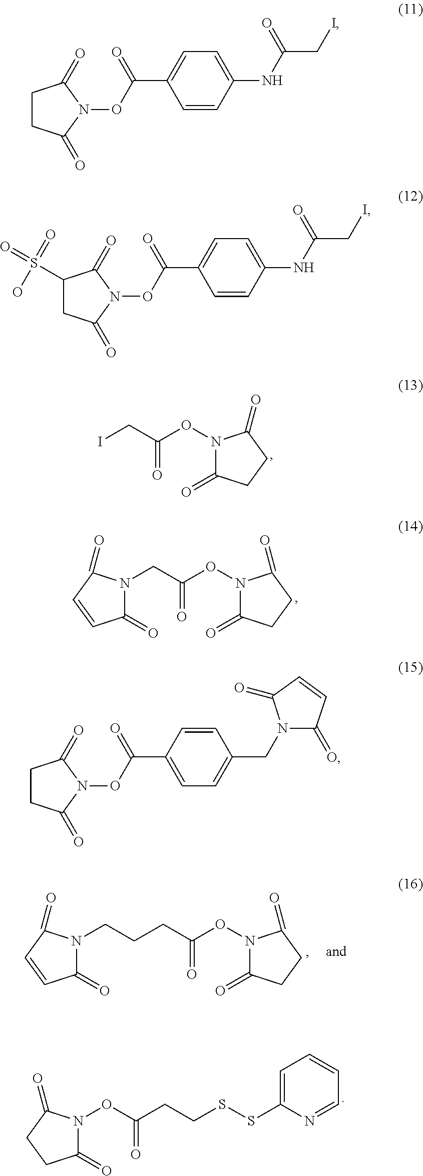

Still referring to FIG. 3, the thiol-and-amine crosslinkers disclosed herein can be used to crosslink a protein substrate with a HECT-ubiquitin ligase to form a crosslinked protein substrate˜HECT-ubiquitin ligase complex. For this representative example, the illustrative HECT-ubiquitin ligase Rsp5 was chosen. The illustrative protein substrate chosen was Sic60-GFP and was tagged with GFP so that in-gel fluorescence imaging could be used to more easily monitor the existence of crosslinking. The ubiquitin ligase Rsp5 has a molecular weight of approximately 92 kDa, and the substrate Sic60-GFP has a molecular weight of approximately 34 kDa. Thus, a band at approximately 130 kDa (that is, the combined molecular weight of the ubiquitin ligase and protein substrate) demonstrates the existence of 1:1 crosslinking ratio between the two molecules to form a crosslinked protein substrate˜HECT-ubiquitin ligase complex that includes a protein substrate˜HECT-ubiquitin ligase pair.

Still referring to FIG. 3, and starting in the upper left-hand corner of the figure, in the first representative experiment between Rsp5 and Sic60-GFP, no thiol-and-amine crosslinker (that is, the absence of any of crosslinkers 10-16) was used. The lack of a band at 130 kDa demonstrates the lack of crosslinking between a protein substrate and ubiquitin ligase in the absence of a thiol-and-amine crosslinker. Now, moving one box to the right, crosslinker 10 was added to the mixture containing Sic60-GFP and Rsp5. In contrast to the previous assay mixture lacking an included thiol-and-amine crosslinker, Sic60-GFP and Rsp5 formed a crosslinked Sic60-GFP˜Rsp5 complex, as demonstrated by the existence of a band at approximately 130 kDa. Still referring to FIG. 3, and now moving through the remainder of the figure, the existence of a 130 kDa band for the remaining mixtures that contain Rsp5 and Sic60-GFP in the presence of crosslinkers 11-16 demonstrates that crosslinked Sic60-GFP˜Rsp5 complexes form from the individual Sic60-GFP and Rsp5 molecules in the presence of the crosslinkers 11-16.

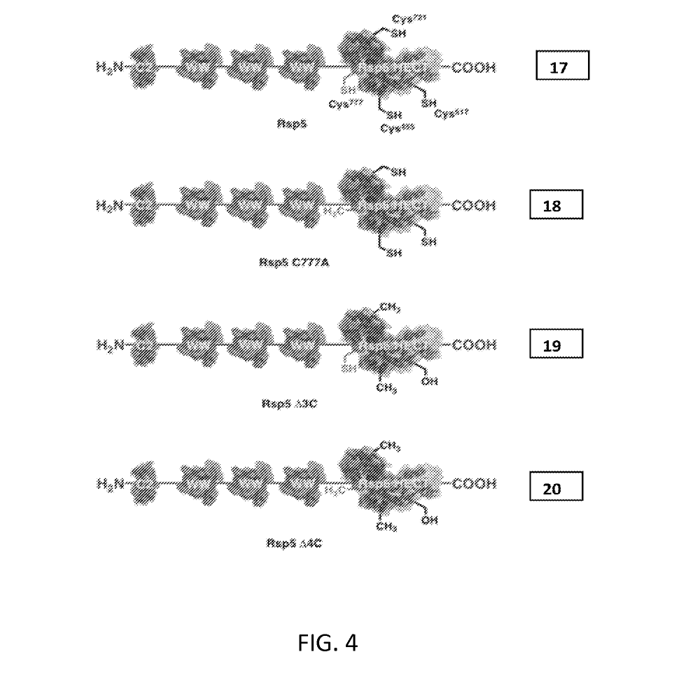

Now referring to FIG. 4, three different types of mutant Rsp5 molecules were prepared to determine whether crosslinking occurred due to the presence of catalytic or surface cysteine sites on the ubiquitin ligase molecule. Species 17 is the normal Rsp5 molecule, which has the full complement of catalytic and surface cysteine sites. Species 18 is a first mutant ubiquitin ligase denoted as C777A, and it has the full complement of surface cysteine sites, but has its catalytic cysteine site removed. Species 19 is a second mutant denoted as Δ3C, and it has all of its surface cysteine sites removed, but retains its catalytic cysteine site. Lastly, species 20 is the final mutant denoted as Δ4C, and it has each of its surface and active cysteine sites removed.

Still referring to FIGS. 3 and 4, a Sic60-GFP˜Rsp5 complex forms between the normal Rsp5 molecule 17 and protein substrate Sic60-GFP in the presence of crosslinkers 10-16. Still referring to FIG. 3, a dramatic decrease in crosslinking is observed when the catalytic cysteine is removed from the ubiquitin ligase, as is demonstrated in the crosslinking interactions for C777A 18 and Δ4C 20. Without being bound or limited to any particular theory governing the mechanism of crosslinker action, one or more residues in or near the catalytic site is one factor for forming a crosslinked complex between a HECT-ubiquitin ligase and protein substrate when using a thiol-and-amine crosslinker, and more specifically with the use of the crosslinkers 10-16. Lastly, still referring to FIGS. 3 and 4, a strong band at 130 kDa is demonstrated for when Δ3C 19 is used in the crosslinking mixture. This observation further supports the view that crosslinked protein substrate˜HECT-ubiquitin ligase complexes preferably result when the catalytic site is used.

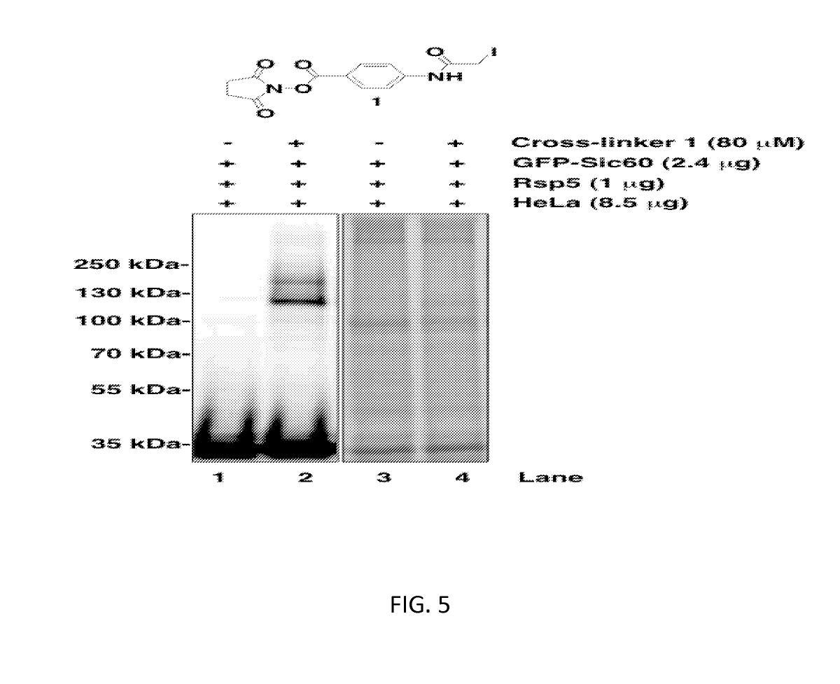

Now referring to FIG. 5, in an alternative embodiment of the crosslinking procedure disclosed herein, the procedure using the aforementioned Rsp5 and Sic60-GFP molecules may be conducted in the presence of cell lysate, which contains the full complement of competing mammalian proteins, many of which possess catalytically active cysteine residues as well as lysines. As demonstrated in FIG. 5, the characteristic band for crosslinking is seen at 130 kDa even in the presence of HeLa cell lysate. Moreover, and still referring to FIG. 5, the data demonstrates that the crosslinking does not occur without the presence of crosslinker, thus demonstrating that the competing mammalian proteins themselves do not lead to crosslinking between the HECT-ubiquitin ligase and protein substrate.

The representative HECT-ubiquitin ligase Rsp5 was chosen to demonstrate the proof-of-concept for the disclosed method, as well as to illustrate the specificity and robustness of the thiol-and-amine crosslinker reagents for an exemplary HECT-ubiquitin ligase and test protein substrate. Without being bound or limited to any particular theory regarding the mechanism of action, one factor in using the crosslinking embodiment disclosed herein is the presence of a catalytic cysteine site on the HECT-ubiquitin ligase of interest.

Thus, in alternative embodiments of the crosslinking interaction disclosed herein, additional HECT-ubiquitin ligases can be used. Such alternative HECT-ubiquitin ligases include yeast HECT ligases, HECTE3 ubiquitin ligases, and HECT-like ubiquitin ligases. Rsp5 is a yeast HECT ligase, and additional yeast HECT ligases within the scope of this disclosure include, but are not limited to: Ufd4; Hu15; Tom1; and Hu14. HECTE3 ubiquitin ligases within the scope of this disclosure include, but are not limited to one of the following: NEDD4; NEDD4L; ITCH; WWP1; WWP2; SMURF1; SMURF2; NEDL1; NEDL2; E6AP; HECTD2; KIAA0614; TRIP12; G2E3; EDD; HACE1; HECTD1; UBE3B; UBEC; KIAA0317; HUWE1; HECTD3; HERC1; HERC2; HERC3; HERC4; HERC5; and HERC6. Lastly, the HECT-like ubiquitin ligases within the scope of this disclosure include, but are not limited to SopA and NIeL.

In additional embodiments disclosed herein, kits for identifying a crosslinked protein substrate˜HECT-ubiquitin ligase complex are disclosed. The first kit disclosed herein includes a thiol-and-amine crosslinker, and a set of instructions for a protocol to use the thiol-and-amine crosslinker to crosslink a protein substrate with a HECT-ubiquitin ligase to form a crosslinked protein substrate˜HECT-ubiquitin ligase complex in a cell lysate, when both the HECT-ubiquitin ligase and protein substrate are endogenous to the cell lysate.

The thiol-and-amine crosslinker in this embodiment may include, but is not limited to, one of the following reagents:

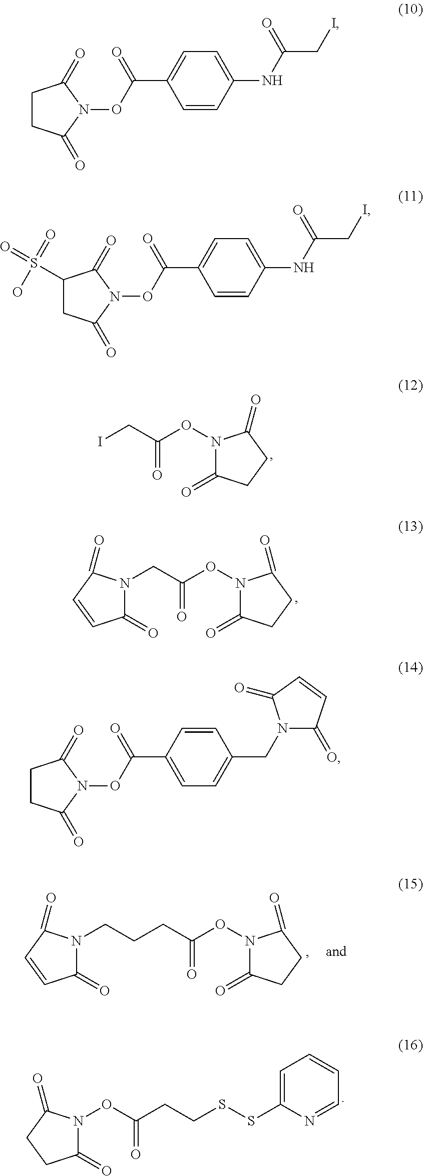

However, the thiol-and-amine crosslinker only needs to have a thiol reactive group and an amine reactive group. The thiol reactive group of the thiol-and-amine crosslinker can include preferably one moiety chosen from the following group:

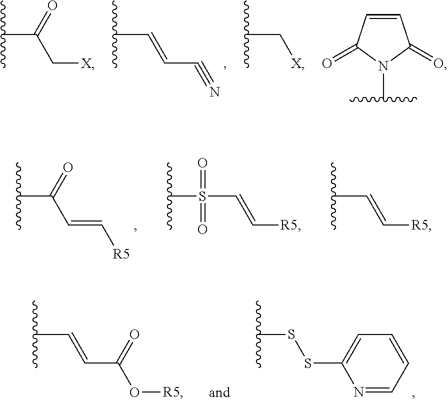

wherein X can be a halogen. Additionally, R5 can be chosen from the group including hydrogen, substituted alkyl, unsubstituted alkyl, substituted heteroalkyl, substituted cycloalkyl, unsubstituted cycloalkyl, substituted heterocycloalkyl, substituted heteroaryl, or unsubstituted heteroaryl.

The amine reactive group of the thiol-and-amine crosslinker can include preferably one moiety chosen from the following group:

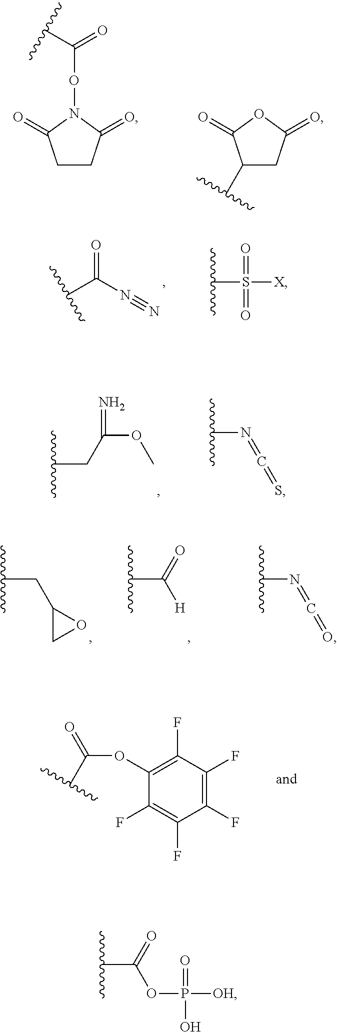

wherein X is a halogen.

In this embodiment, the kit may be used to crosslink endogenous HECTE3 ubiquitin ligases including, but not limited to, one of the following: NEDD4; NEDD4L; ITCH; WWP1; WWP2; SMURF1; SMURF2; NEDL1; NEDL2; E6AP; HECTD2; KIAA0614; TRIP12; G2E3; EDD; HACE1; HECTD1; UBE3B; UBEC; KIAA0317; HUWE1; HECTD3; HERC1; HERC2; HERC3; HERC4; HERC5; and HERC6.

A second kit disclosed herein includes a buffer solution, a thiol-and-amine crosslinker, and a set of instructions for a protocol to use the thiol-and-amine crosslinker to crosslink a protein substrate with a HECT-ubiquitin ligase resulting in a crosslinked protein substrate˜HECT-ubiquitin ligase complex in the provided buffer solution.

In a first aspect of this kit, the buffer solution includes mammalian cell lysate. In an additional aspect, the buffer solution includes a HECT-ubiquitin ligase. The HECT-ubiquitin ligase may be a yeast HECT ligase, a HECT-like ubiquitin ligase, or a HECTE3 ubiquitin ligase. Yeast HECT ligases that may be incorporated include, but are not limited to: Rsp5; Ufd4; Hu15; Tom1; and Hu14. HECT-like ubiquitin ligases that may be incorporated include, but are not limited to SopA and NIeL. The HECTE3 ubiquitin ligases that may be incorporated include, but are not limited to: NEDD4; NEDD4L; ITCH; WWP1; WWP2; SMURF1; SMURF2; NEDL1; NEDL2; E6AP; HECTD2; KIAA0614; TRIP12; G2E3; EDD; HACE1; HECTD1; UBE3B; UBEC; KIAA0317; HUWE1; HECTD3; HERC1; HERC2; HERC3; HERC4; HERC5; and HERC6.

In this kit embodiment, the thiol-and-amine crosslinker in the kit may include, but is not limited to, one of the following:

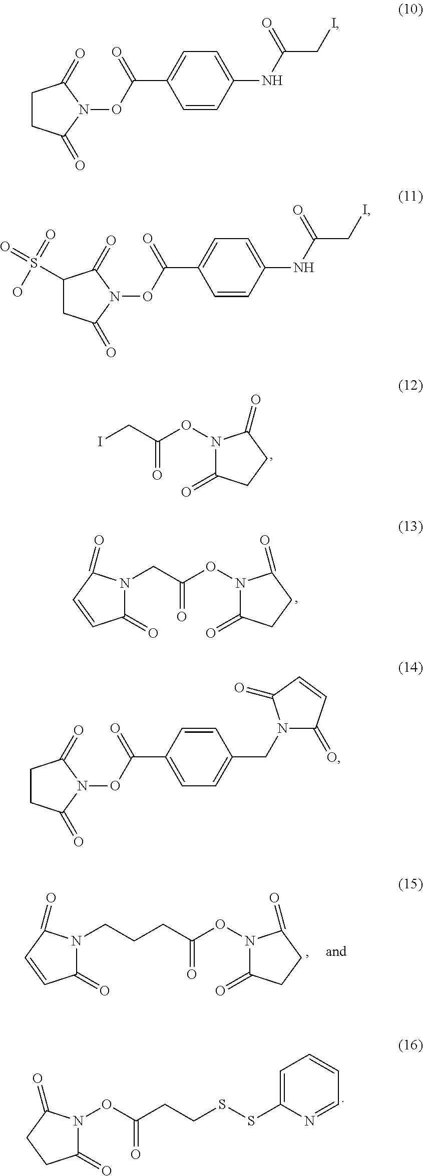

However, the thiol-and-amine crosslinker only needs to have a thiol reactive group and an amine reactive group. The thiol reactive group of the thiol-and-amine crosslinker can include preferably one moiety chosen from the following group:

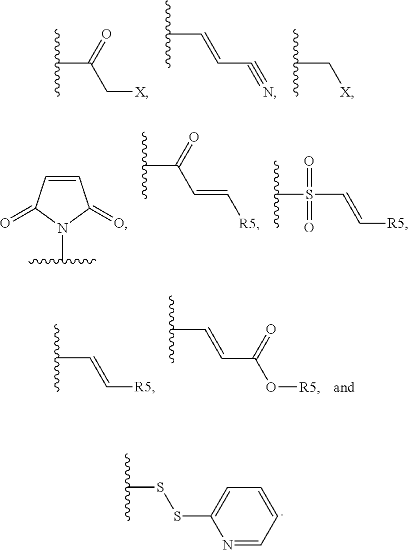

wherein X can be a halogen. Additionally, R5 can be chosen from the group including hydrogen, substituted alkyl, unsubstituted alkyl, substituted heteroalkyl, substituted cycloalkyl, unsubstituted cycloalkyl, substituted heterocycloalkyl, substituted heteroaryl, or unsubstituted heteroaryl.

The amine reactive group of the thiol-and-amine crosslinker can include preferably one moiety chosen from the following group:

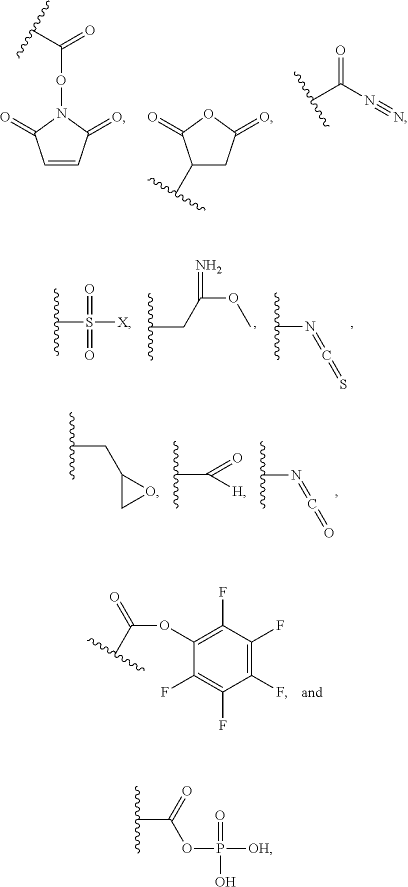

wherein X is a halogen.

These methods also provide a robust and general means for identifying a protein substrate for a HECT-ubiquitin ligase. The method includes providing a sample suspected to contain a protein substrate for a HECT-ubiquitin ligase; adding a buffer solution to the sample to form a first mixture; adding a thiol-and-amine crosslinker to the first mixture to create a second mixture; reacting the second mixture under conditions to create a crosslinked protein substrate HECT-ubiquitin ligase pair; and adding a quenching solution to the mixture. The presence of the crosslinked protein substrate HECT-ubiquitin ligase pair is indicative of a protein substrate for a HECT-ubiquitin ligase.

The crosslinked protein substrate˜HECT-ubiquitin ligase complex may be isolated using a variety of techniques, including the use of affinity-tag reagents that retrieve pre-labeled HECT-ubiquitin ligase containing a tag (for example, hexahistidine motif, a GST moiety, a biotin moiety, etc.) or use of an anti-HECT-ubiquitin ligase antibody in conjunction with immunoprecipitation or affinity chromatography methods. Such HECT-ubiquitin ligase reagents can be generated readily by molecular biological, genetic and immunological approaches, all of which are known in the art or that may be available from commercial sources. The use of a reversible or cleavable thiol-and-amine crosslinker is preferred in such assays to enable release of the protein substrate from the HECT-ubiquitin ligase in the crosslinked protein substrate˜HECT-ubiquitin ligase complex. The released protein substrate can then be identified based upon limited protein sequencing analyses of the protein, coupled to molecular biology and recombinant, molecular genetic approaches as well as proteomic and genomic database searching tools. This method may enable for rapid identification of proteins important for use as reagents and markers for various purposes, including without limitation, uses for diagnostic and therapeutic applications.

### Examples

### Reagents and Materials

UBE1(yeast) and UbcH5a (human recombinant) were purchased from R&D Systems. Ubiquitin (from bovine erythrocytes) was purchased from Sigma-Aldrich. All bifunctional, thiol-and-amine crosslinkers were purchased from Pierce Biotechnology (Rockford, Ill.). Such bifunctional crosslinking reagents are also available from other commercial sources including the following: Calbiochem (San Diego, Calif.); G-Biosciences (St. Louis, Mo.); Life Technologies (Grand Island, N.Y.); ProteoChem, Inc. (Denver, Colo.); PrimeTech (Minsk, Belarus); and Biomol GmbH (Hamburg, Del.). The purchased proteins were used without further purification. Precast 12% and 7.5% SDS gels were purchased from Biorad (Mini-PROTEAN precast gels). In-gel fluorescence imaging was performed on a Typhoon 9600 (GE Healthcare). All gels were visualized with the Colloidal Blue Staining Kit (Invitrogen). GST-Rsp5 in pGEX-6p-1 and Sic60-GFP in pET3a vectors were gifts from Prof. Andreas Matouschek, and Rsp5ΔWW in pGEX-6p-1 was a gift from Prof. Linda Hicke, both of Northwestern University. For MS/MS analysis, excised protein bands of interest were reduced by DTT, alkylated with iodoacetamide, and then digested with trypsin. The extracted peptides were analyzed by nano-capillary LC-MS using a 100 mm×75 μm C₁₈ column in-line with a 7T LTQ-FT (ThermoFisher, San Jose, Calif.).

### Example 1

### Purification of Rsp5 and Mutant Forms

BL21(DE3)pLysS cells (Novagen) were transformed with GST-Rsp5 in pGEX-6p-1 vector, and were induced to express with IPTG (0.5 mM). Induction of GST-Rsp5 was performed at 18° C. overnight. Cells were then harvested and lysed by sonication in phosphate-buffered saline (PBS) with protease inhibitors (Complete Mini Protease Inhibitor Cocktail, Roche). The supernatant was incubated with glutathione agarose beads (Pierce Biotechnology) for 1-2 hr at 4° C. The beads were washed three times with PBS and incubated with PreScission Protease overnight at 4° C. to elute Rsp5 (50 mM HEPES, 150 mM NaCl, 0.1 mM EDTA). Rsp5 C777A, Rsp5 Δ3C, Rsp5 Δ4C mutant proteins were prepared using the same protocol. Mutations of surface and catalytic cysteines in Rsp5 were performed with a QuickChange kit (Stratagene).

### Example 2

### Purification of Sic60-GFP and Mutant Forms

BL21(DE3)pLysS cells (Novagen) were transformed with Sic60-GFP in pET3a vector, and were induced to express with IPTG (1.0 mM). Induction of Sic60-GFP was performed at 37° C. for 4 hr. Cells were then harvested and lysed by sonication in Buffer A (50 mM NaPO₄, 300 mM NaCl, pH 7) with protease inhibitors (Complete Mini Protease Inhibitor Cocktail, EDTA free, Roche). The resulting supernatant was incubated with HisPur Ni-NTA Resin (Pierce Biotechnology) at 4° C. for 1-2 hr. Beads were then washed with PBS, Buffer B (50 mM NaPO₄, 300 mM NaCl, 10 mM imidazole, pH 7) prior to eluting protein with Buffer C (50 mM NaPO₄, 300 mM NaCl, 150 mM imidazole, pH 7). The elution fraction was dialyzed against buffer D (50 mM HEPES, 150 mM NaCl, 0.1 mM EDTA) overnight.

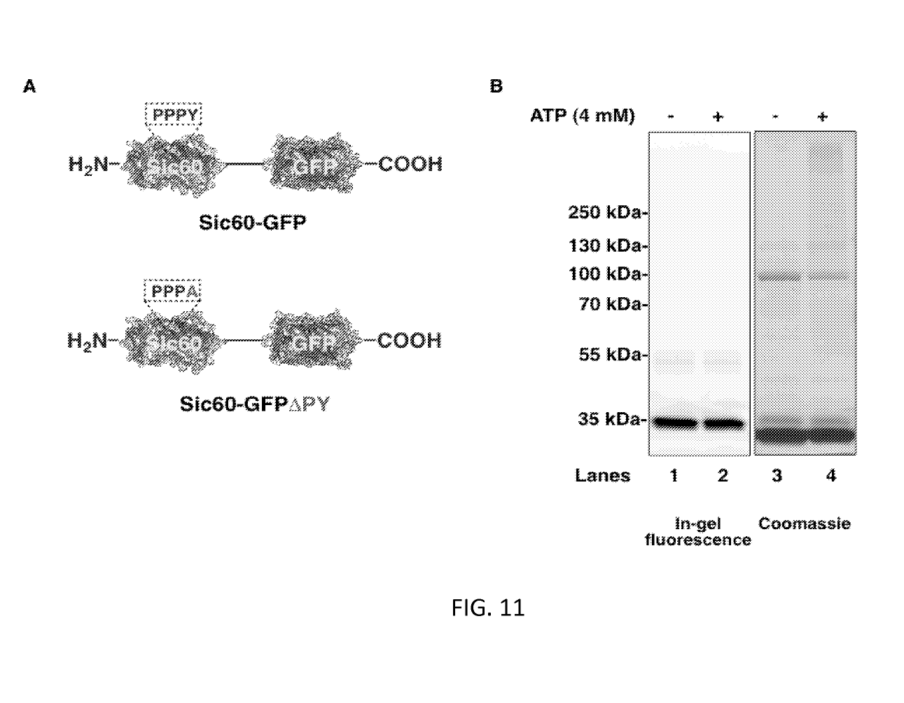

Sic60-GFPΔPY mutant protein was prepared using the same protocol. Mutations of Tyr⁹ to Ala⁹ residues in Sic60-GFP were performed with a QuickChange kit (Stratagene). An illustration of this mutant protein is depicted in FIG. 11A and the ubiquitination activity using this mutant protein as a substrate is illustrated in FIG. 11B.

### Example 3

### Enzymatic Activity Assay for Rsp5, Rsp5 C777A, Rsp5 Δ3C, and Rsp5Δ4C

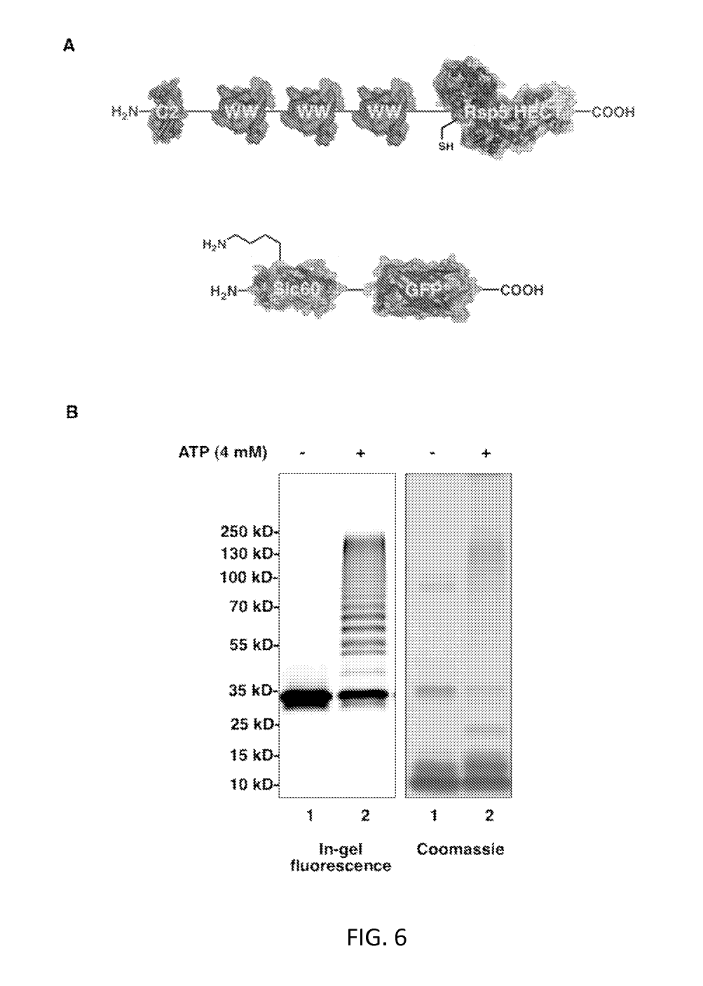

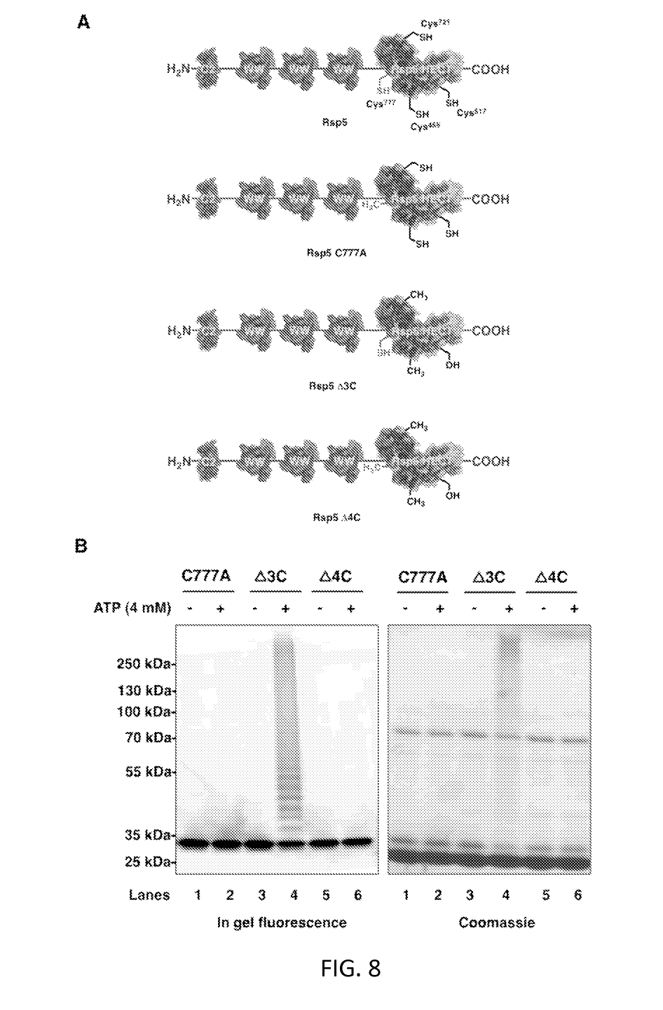

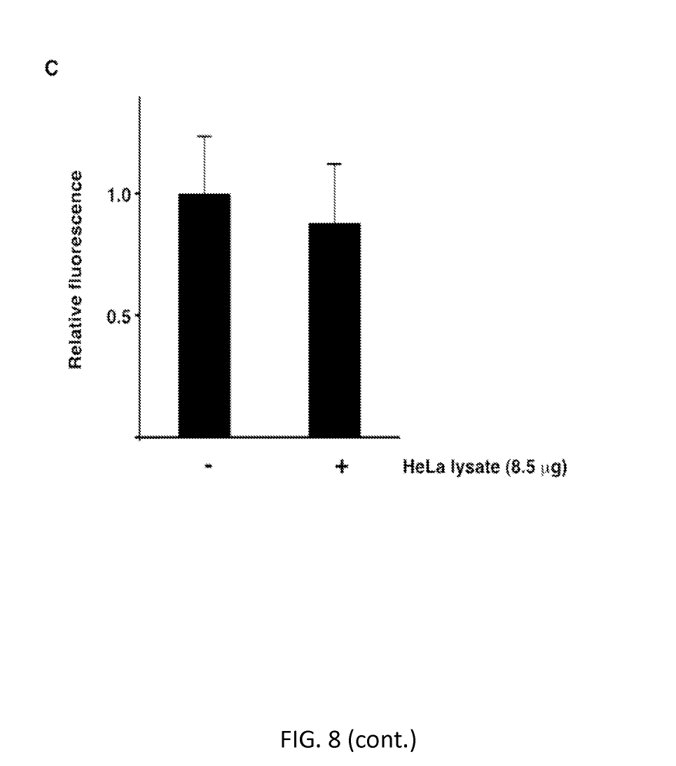

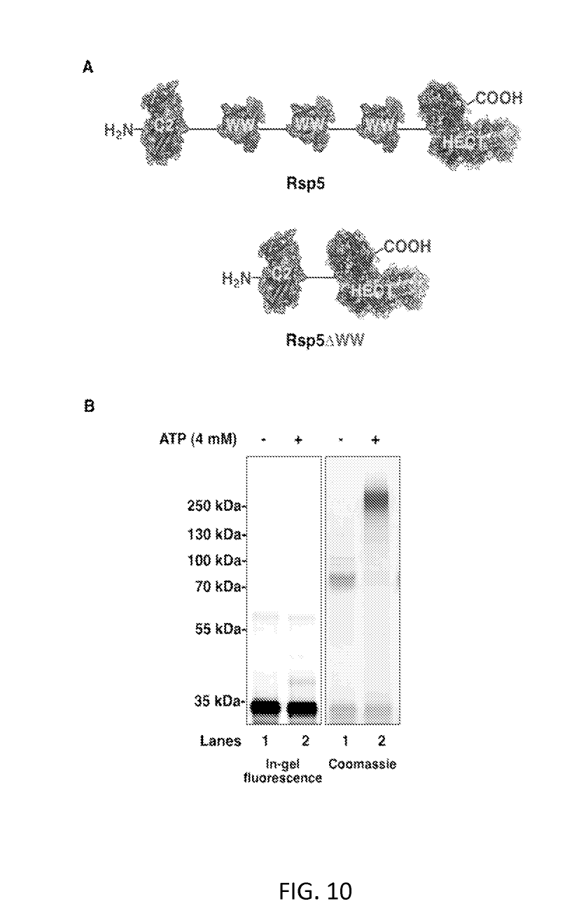

All enzymatic reactions (30 μL total volume) were performed in buffer containing HEPES (25 mM. pH 7.6), NaCl (50 mM), MgCl₂ (4 mM) with the indicated amount of UBE1, UbcH5a, ubiquitin, Rsp5 and substrates. Upon addition of ATP (4 mM), reactions were incubated for 2 hr and were then quenched with 6 μL of 6x Laemmli loading buffer, resolved by 12% or 7.5% SDS-PAGE, and imaged by in-gel scanning fluorescence imaging (Typhoon 9600, GE Healthcare). In the negative control reactions, the corresponding volume of water was added instead of ATP. Colloidal Coomassie staining reagent was also used to visualize ubiquitinated proteins. For examples of these assays, see FIGS. 6B, 8B, 10B and 11B.

### Example 4

### Crosslinking Assay

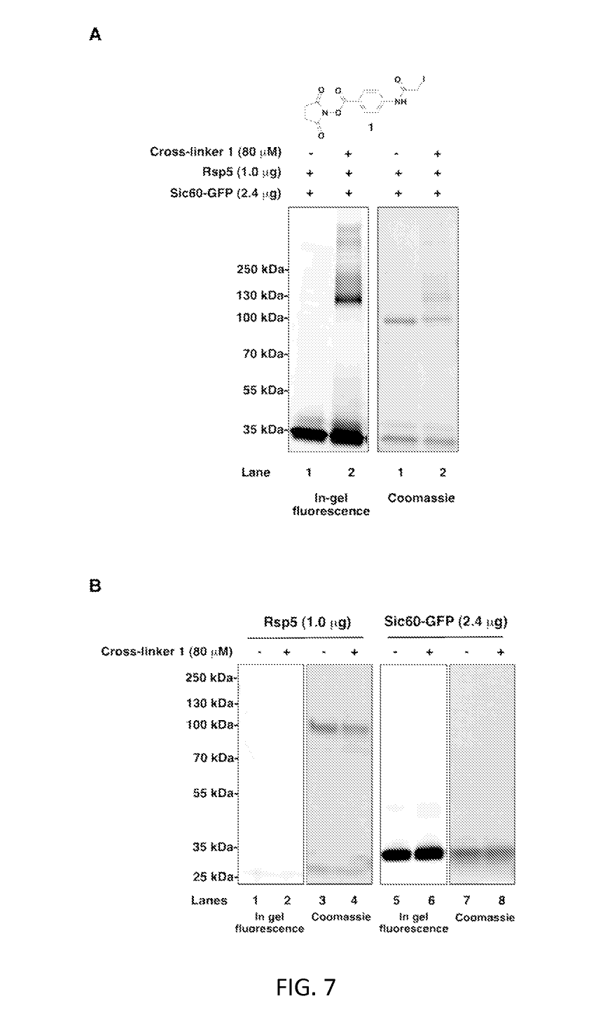

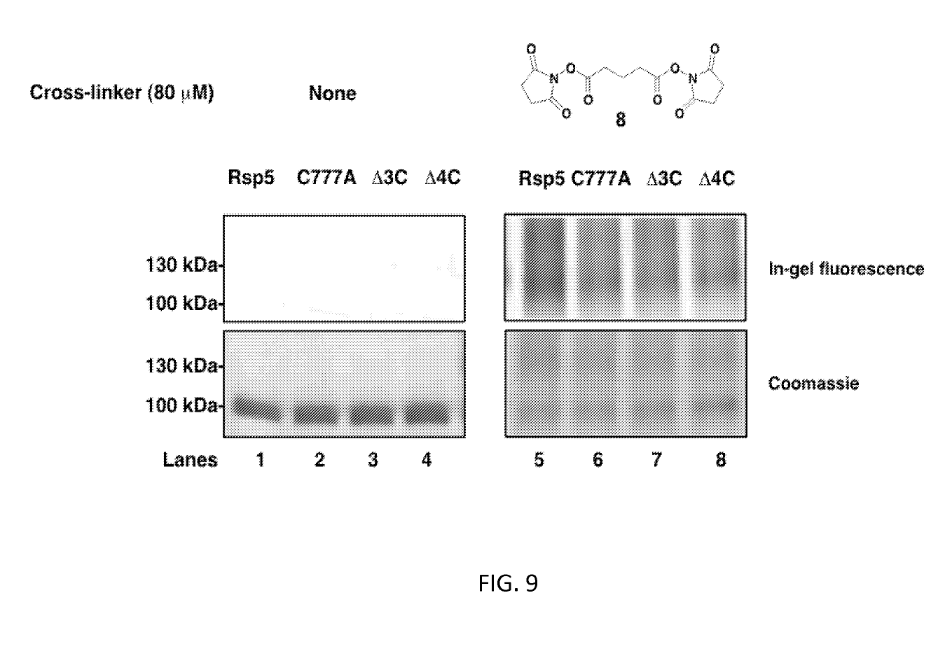

All crosslinking reactions (30 μL total volume) were performed in buffer that contains HEPES (25 mM, pH 7.6), NaCl (50 mM), MgCl₂ (4 mM) and Triton X (1.0%) with indicated amounts of Rsp5 and substrate. Crosslinking reactions were initiated by adding 0.24 μL of a thiol-and-amine crosslinking agent stock solution (10 mM thiol-and-amine crosslinking agent in DMSO) to the reaction mixture. After incubating the reaction mixture at room temperature for 1 hr, reactions were quenched by adding 6 μL of 6x Laemmli loading buffer, 1 μL of β-mercaptoethanol and 1.5 μL of 1 M Tris buffer (pH 7.6). For the reductively cleavable crosslinker 16, β-mercaptoethanol was not used in the quench. The sample mixtures (12.0 μL total) were resolved by 7.5% SDS-PAGE and imaged by in-gel scanning fluorescence (Typhoon 9600, GE Healthcare). In the negative control reactions, the corresponding volume of DMSO was added instead of the crosslinkers. Colloidal Coomassie staining reagent was also used to visualize crosslinked proteins. Examples of the crosslinking assays are illustrated, for example, in FIGS. 3, 5, 7, 8C and 9.

### Example 5

### Purification of Intact GST-Rsp5 HECT-Ubiquitin Ligase (Prophetic)

GST-Rsp5 HECT-ubiquitin ligase will be purified from bacterial expression lysates with the GST tag intact on the recombinant GST-Rsp5 HECT-ubiquitin ligase using the following procedure. Bacterial expression lysate containing recombinant GST-Rsp5 HECT-ubiquitin ligase will be incubated with glutathione agarose beads (Pierce Biotechnology) at 4° C. for 1-2 hr. The beads will be washed three times with buffer (50 mM Tris, 150 mM NaCl, pH 8.0) and subjected to chromatography using a gradient of 1 mM to 50 mM reduced glutathione in buffer (50 mM Tris, 150 mM NaCl, pH 8.0) at 4° C. to elute the GST-Rsp5 HECT-ubiquitin ligase. The GST-Rsp5 HECT-ubiquitin ligase will be subjected to dialysis and concentration against glutathione-free buffer to remove the glutathione.

### Example 6

### Identifying Protein Substrates for a HECT-Ubiquitin Ligase (Prophetic)

Two different procedures are presented that use different thiol-and-amine crosslinkers to capture the protein substrates of a HECT-ubiquitin ligase. In the first procedure, an acid-labile thiol-and-amine crosslinker will be used. In the second procedure, a thiol-cleavable thiol-and-amine crosslinker will be used.

Procedure 1:

A volume (100 μL) of clarified yeast cellular extract containing 100 μg of whole cell yeast protein will be introduced into a 1.5 mL microfuge tube. This volume will be adjusted with a concentrated buffer solution to provide a final buffer solution consisting of HEPES (25 mM. pH 7.6), NaCl (50 mM), MgCl₂ (4 mM) and Triton X (1.0%) to form a first mixture. A 10 μL volume of recombinant GST-Rsp5 HECT-ubiquitin ligase (1 mg/mL), which will be prepared according to Example 5, will be added to the first mixture to form a second mixture. After incubating the second mixture at a temperature ranging from 4° C. to 30° C. for 5-10 min, a volume (10 μL) of 25 mM Succinimidyl 3-(bromoacetamido)-propionate (Pierce/Thermo Scientific Catalog #22339) will be added to the second mixture to form the crosslinking reaction mixture. After incubating the crosslinking reaction mixture at room temperature for 1 hr, the crosslinking reaction will be quenched by adding 10 μL of 50 mM β-mercaptoethanol and 10 μL of 1 M Tris buffer (pH 7.6) to the crosslinking reaction mixture to form a quenched reaction mixture. The quenched reaction mixture will be incubated at room temperature for 10 min.

The crosslinked protein substrate˜GST-Rsp5 complex(es) will be purified from the quenched reaction mixture by incubating the quenched reaction mixture with glutathione agarose beads (Pierce Biotechnology) at 4° C. for 1-2 hr. The beads will be washed three times with PBS to remove any uncrosslinked proteins.

The crosslinked protein substrate˜GST-Rsp5 HECT-ubiquitin ligase complex(es) bound to the glutathione agarose beads will be subjected to mild acid hydrolysis to cleave the crosslinker. The initial supernatant from this mild acid treatment and subsequent, mildly acidic washes of the glutathione agarose beads will contain released protein substrate(s) free of the recombinant GST-Rsp5 HECT-ubiquitin ligase. The initial and subsequent wash supernatants will be combined and neutralized with appropriately pH buffer at a suitable concentration.

Procedure 2:

The same methods will be used for the second procedure as described for the first procedure, except for three differences in method details. First, the second procedure will use a volume (10 μL) of 25 mM thiol-and-amine crosslinker 16 in place of the Succinimidyl 3-(bromoacetamido)propionate (Pierce/Thermo Scientific Catalog #22339) that is described in the first procedure. Second, the second procedure will use a quenching solution lacking β-mercaptoethanol, which is present in the quenching solution of the first procedure. This is an important modification because the thiol-and-amine crosslinker 16 contains a disulfide bond that is sensitive to cleavage by β-mercaptoethanol. So inclusion of β-mercaptoethanol prior to purification of the crosslinked protein substrate˜GST-Rsp5 HECT-ubiquitin ligase complexes would result in premature loss of the crosslinked protein substrate(s) before purification of the complexes. Third, the second procedure will use β-mercaptoethanol (10 mM -100 mM) rather than acid to cleave the crosslinker, thereby releasing the protein substrate(s) from the crosslinked protein substrate˜GST-Rsp5 HECT-ubiquitin ligase complexes following their purification (that is, when the complexes are immobilized on the glutathione column).

The solution containing released protein substrate(s) from foregoing procedures will be concentrated or precipitated using standard methods. The released protein substrate(s) will be resuspended in loading buffer and chromatographically resolved by gel electrophoresis on SDS-PAGE gels. Following silver staining of the gels after electrophoresis, bands corresponding to individual protein species will be excised for protein sequence analyses according to standard techniques. The identity of the protein species will be deduced based upon a combination of protein sizing, protein sequence analysis, as well as genomic and proteomic bioinformatics. These proteins may then be selected for expression and use as reagents for diagnostic and/or therapeutic applications.

It should be understood that the methods, procedures, operations, devices, and systems illustrated in the figures may be modified without departing from the spirit of the present disclosure. For example, these methods, procedures, operations, devices and systems may include more or fewer steps or components than appear herein, and these steps or components may be combined with one another, in part or in whole.

Furthermore, the present disclosure is not to be limited in terms of the particular embodiments described in this application, which are intended as illustrations of various aspects. Many modifications and variations can be made without departing from its scope and spirit. Functionally equivalent methods and apparatuses within the scope of the disclosure, in addition to those enumerated herein, will be apparent to those skilled in the art from the foregoing descriptions.

### TERMINOLOGY AND DEFINITIONS

The terminology used herein is for the purpose of describing particular embodiments only, and is not intended to be limiting. With respect to the use of substantially, any plural and/or singular terms herein, those having skill in the art can translate from the plural as is appropriate to the context and/or application. The various singular/plural permutations may be expressly set forth herein for the sake of clarity.

Terms used herein are intended as “open” terms. For example, the term “including” also means “including but not limited to;” the term “having” also means “having at least;” and the term “includes” also means “includes but is not limited to.”

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, includes the number recited and refer to ranges which can subsequently be broken down into sub-ranges as discussed above.

A range includes each individual member. Thus, for example, a group having 1-3 members refers to groups having 1, 2, or 3 members. Similarly, a group having 5 members refers to groups having 1, 2, 3, 4, or 5 members, and so forth.

The phrase “thiol-and-amine crosslinker” refers to a crosslinking regent having at least one thiol-reactive group and at least one amine-reactive group. Preferred thiol-and-amine crosslinkers are bifunctional crosslinking reagents having one thiol-reactive group and one amine-reactive group.

As used herein, the phrase “crosslinked protein substrate˜HECT-ubiquitin ligase complex” refers to at least one covalent species that contains at least one protein substrate for a HECT-ubiquitin ligase and at least one HECT-ubiquitin ligase, wherein the at least one protein substrate for a HECT-ubiquitin ligase is covalently-coupled to the at least one HECT-ubiquitin ligase through reaction with at least one bifunctional thiol-and-amine crosslinker. As used herein, “crosslinked protein substrate˜HECT-ubiquitin ligase complex” includes a protein substrate˜HECT-ubiquitin ligase pair formed through reaction between one protein substrate for a HECT-ubiquitin ligase and one HECT-ubiquitin ligase with at least one bifunctional thiol-and-amine crosslinker. Finally, “crosslinked protein substrate˜HECT-ubiquitin ligase complex” also includes higher order covalent species that contain more than one protein substrate for a HECT-ubiquitin ligase and/or more than one HECT-ubiquitin ligase coupled together through reaction with one or more bifunctional thiol-and-amine crosslinkers.

The phrase “protein substrate˜HECT-ubiquitin ligase pair” refers to a complex between one protein substrate for a HECT-ubiquitin ligase and one HECT-ubiquitin ligase that are covalently-coupled together with at least one bifunctional thiol-and-amine crosslinker.

Several terms having the same meaning are used interchangeably as described herein. The following pairs of terms have the same meaning with respect to that pair: Rsp5 C777A and C777A; Rsp5 Δ3C and Δ3C; Rsp Δ4C and Δ4C; and GFP-Sic60 and Sic60-GFP.

What is claimed is:

1. A method for forming a crosslinked protein substrate˜HECT-ubiquitin ligase complex in a buffer solution, comprising:
providing a buffer solution;
adding a thiol-and-amine crosslinker to the buffer solution to create a mixture;
and
reacting the mixture to create a crosslinked protein substrate˜HECT-ubiquitin ligase complex.

2. The method of claim 1, wherein the buffer solution comprises a mammalian cell lysate.

3. The method of claim 1 or 2, wherein the buffer solution comprises at least one HECT-ubiquitin ligase.

4. The method of claim 3, wherein the at least one HECT-ubiquitin ligase comprises a HECTE3 ubiquitin ligase.

5. The method of claim 4, wherein the HECTE3 ubiquitin ligase is selected from the group consisting of NEDD4L, ITCH, WWP1, WWP2, SMURF1, SMURF2, NEDL1, NEDL2, E6AP, HECTD2, KIAA0614, TRIP12, G2E3, EDD, HACE1, HECTD1, UBE3B, UBEC, KIAA0317, HUWE1, HECTD3, HERC1, HERC2, HERC3, HERC4, HERC5 and HERC6.

6. The method of claim 3, where the at least one HECT-ubiquitin ligase comprises a HECT-like ligase.

7. The method of claim 6, wherein the HECT-like ligase is SopA or NIeL.

8. The method of claim 3, wherein the at least one HECT-ubiquitin ligase comprises a yeast HECT ligase.

9. The method of claim 8, wherein the yeast HECT ligase is selected from the group consisting of Rsp5, Ufd4, Hu15, Tom1 and Hu14.

10. The method of any of the preceding claims, wherein the buffer solution comprises a protein substrate of at least one HECT-ubiquitin ligase.

11. The method of any of the preceding claims, wherein the crosslinked protein substrate˜HECT-ubiquitin ligase complex comprises a crosslinked protein substrate˜HECT-ubiquitin ligase pair.

12. The method of the preceding claims, wherein the thiol-and-amine crosslinker is selected from the group consisting of:

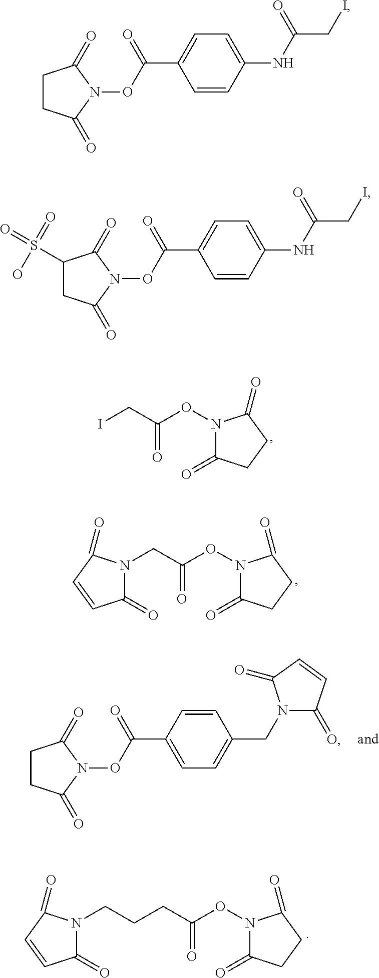 [SMILES: O=C(CI)Nc1ccc(C(=O)ON2C(=O)CCC2=O)cc1 ; O=C(CI)Nc1ccc(C(=O)ON2C(=O)CC(S(=O)(=O)O)C2=O)cc1 ; O=C(CI)ON1C(=O)CCC1=O ; O=C(CN1C(=O)C=CC1=O)ON1C(=O)CCC1=O ; O=C(ON1C(=O)CCC1=O)c1ccc(CN2C(=O)C=CC2=O)cc1 ; O=C(CCCN1C(=O)C=CC1=O)ON1C(=O)CCC1=O]

13. The method of any of claims 1-11, wherein the thiol-and-amine crosslinker is:

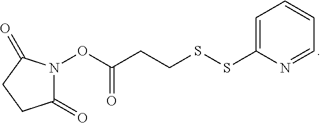 [SMILES: O=C(CCSSc1ccccn1)ON1C(=O)CCC1=O]

14. The method of claim 1, further comprising a step of adding a quenching solution to the mixture.

15. A method of claim 14, wherein the quenching solution comprises a loading buffer, β-mercaptoethanol and tris buffer.

16. A method of claim 14, wherein the quenching solution comprises of loading buffer and tris buffer.

17. A kit for forming a crosslinked protein substrate˜HECT-ubiquitin ligase complex in a cell lysate, comprising:
a thiol-and-amine crosslinker; and
instructions to use the thiol-and-amine crosslinker in a cell lysate to crosslink an endogenous protein substrate with an endogenous HECT-ubiquitin ligase resulting in the formation of a crosslinked protein substrate˜HECT-ubiquitin ligase complex.

18. The kit of claim 17, wherein the thiol-and-amine crosslinker is selected from the group consisting of

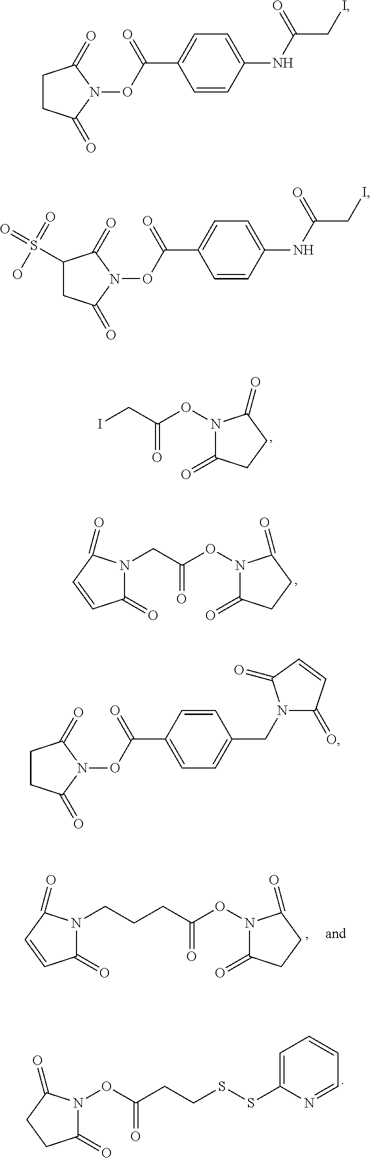

19. The kit of claim 17 or 18, wherein the crosslinked protein substrate˜HECT-ubiquitin ligase complex comprises a crosslinked protein substrate˜HECT-ubiquitin ligase pair.

20. A kit for forming a crosslinked protein substrate˜HECT-ubiquitin ligase complex in buffer solution, comprising:
a buffer solution;
a thiol-and-amine crosslinker; and
instructions to use said thiol-and-amine crosslinker to crosslink a protein substrate with a HECT-ubiquitin ligase in said buffer solution resulting in the formation of a crosslinked protein substrate˜HECT-ubiquitin ligase complex.

21. The kit of claim 20, wherein the crosslinked protein substrate˜HECT-ubiquitin ligase complex comprises a crosslinked protein substrate˜HECT-ubiquitin ligase pair.

22. The kit of claims 20 and 21, wherein the buffer solution comprises a mammalian cell lysate.

23. The kit of any one of claims 20-22, wherein the buffer solution comprises at least one HECT-ubiquitin ligase.

24. The kit of claim 23, wherein the at least one HECT-ubiquitin ligase comprises a HECTE3 ubiquitin ligase.

25. The kit of claim 24, wherein the HECTE3 ubiquitin ligase is selected from the group consisting of NEDD4, NEDD4L, ITCH, WWP1, WWP2, SMURF1, SMURF2, NEDL1, NEDL2, E6AP, HECTD2, KIAA0614, TRIP12, G2E3, EDD, HACE1, HECTD1, UBE3B, UBEC, KIAA0317, HUWE1, HECTD3, HERC1, HERC2, HERC3, HERC4, HERC5 and HERC6.

26. The kit of claim 23, wherein the at least one HECT-ubiquitin ligase comprises a HECT-like ubiquitin ligase.

27. The kit of claim 26, wherein the HECT-like ubiquitin ligase is SopA or NIeL.

28. The kit of claim 23, wherein the at least one HECT-ubiquitin ligase comprises a yeast HECT ligase.

29. The kit of claim 28, wherein the yeast HECT ligase is selected from the group consisting of Rsp5, Ufd4, Hu15, Tom1 and Hu14.

30. The kit of any one of claims 20-29, wherein the thiol-and-amine crosslinker is selected from the group consisting of: [chemical structure]
